(19) United States
(12) Patent Application Publication
(10) Pub. No.: US 2021/0205134 A1
(43) Pub. Date: Jul. 8, 2021

(54) METHOD AND SYSTEM FOR ADJUSTING THE REFRACTIVE POWER OF AN IMPLANTED INTRAOCULAR LENS

(71) Applicant: LensGen, Inc., Irvine, CA (US)

(72) Inventors: Ramgopal Rao, Irvine, CA (US); Thomas Silvestrini, Alamo, CA (US)

(21) Appl. No.: 17/147,820

(22) Filed: Jan. 13, 2021

Related U.S. Application Data
(63) Continuation of application No. 15/901,595, filed on Feb. 21, 2018, now abandoned.
Application No. 15/901,595 is a continuation of application No. 14/397,567, filed on Oct. 28, 2014, now abandoned.
(60) Provisional application No. 61/640,518, filed on Apr. 30, 2012.

(51) Int. Cl. A61F 9/008; A61F 2/16

(52) U.S. Cl. CPC A61F 9/00812; A61F 2/1624; A61F 9/00834; A61F 9/00838; A61F 2009/00885; A61F 2250/0003; A61F 2009/0087; A61F 2009/00887; A61F 2/16

(57) ABSTRACT

A method for adjusting the refractive power of a fluid-filled intraocular lens implanted into a patients eye. The method comprises selecting a pattern to cause a flattening of the intraocular lens or an increase in curvature of the intraocular lens, and ablating the pattern, onto either an optical element of the intraocular lens or a flexible element of the intraocular lens, to alter either one or both of a refractive power and an amplitude of accommodation of the intraocular lens. The ablating occurs while the intraocular lens remains implanted in the patient's eye. The ablating maintains the integrity of a fluid-filled interior cavity defined between the optical element and the flexible element, but causes the flattening of the intraocular lens or the increase in curvature of the intraocular lens.

### CROSS-REFERENCE TO RELATED APPLICATION

The present application claims priority to U.S. patent application Ser. No. 14/397,567, filed Oct. 28, 2014, which is a national stage application pursuant to 35 U.S.C. § 371 of International Application No. PCT/US2013/038943, filed Apr. 30, 2013, which claims priority to U.S. Provisional Application No. 61/640,518, filed Apr. 30, 2012, the entire contents of which are incorporated into this application by reference.

### FIELD OF THE INVENTION

The present invention relates generally to intraocular lens devices and, more particularly, to systems and methods for post-operatively changing and/or adjusting the refractive power of an intraocular lens by a laser.

### BACKGROUND

Cataract surgery and intraocular lens (IOL) implantation are one of the most commonly performed surgeries in the world. The objective of the surgery is that the implanted IOL will achieve complete correction of cumulative refractive error of the eye undergoing surgery. However, various confounding factors such as errors in geometrical measurement of the eye, post-surgical changes in the lens position and unexpected anatomical features of an eye may induce post-surgical refractive errors. New classes of IOLs that have the ability to change the refractive power of the lens on demand are now commercially available, such as multifocal lenses, pseudo-accommodative lenses, or accommodative lenses. For various reasons, a vast majority of these lenses achieve only a limited range (amplitude) of accommodation, which is less than satisfactory.

There is therefore a need for an intraocular lens that provides for a greater amplitude of accommodation.

### SUMMARY OF THE INVENTION

The invention is directed to systems and methods for changing and/or adjusting the refractive power of an intraocular lens by a laser. The system and method disclosed herein allow for the refractive power of the intraocular lens to be changed post-operatively, after implantation of the intraocular lens in a patients eye.

The present invention is embodied in a method for post-operatively adjusting the refractive power of an intraocular lens implanted into a patient's eye. The method comprises ablating a surface of the intraocular lens to change either one or both of a refractive power and an amplitude of accommodation of the intraocular lens, wherein the step of ablating a surface of the intraocular lens occurs while the intraocular lens remains implanted in the patient's eye.

In a first aspect of this embodiment, the surface of the intraocular lens is ablated by a laser. The laser may be a femtosecond laser or a YAG laser.

In a second aspect of this embodiment, the surface of the intraocular lens may be ablated to thin the intraocular lens to provide a greater amplitude of accommodation of the intraocular lens.

In a third aspect of this embodiment, the laser may be used to ablate a pattern onto the surface of the intraocular lens. In a further aspect, the pattern may comprise a circular region of the surface of the intraocular lens. Alternatively, the pattern may comprise a ring-shaped region of the surface of the intraocular lens. In yet another aspect, the pattern may comprise arcuate ablations. The arcuate ablations may correct an astigmatism on the patient's eye.

In a fourth aspect of this embodiment, the pattern may be selected to cause a flattening of the intraocular lens. Alternatively, the pattern may be selected to cause an increase in curvature of the intraocular lens.

In a fifth aspect of this embodiment, the ablating may be performed within an optical axis of the patient's eye. Alternatively, the ablating may be performed entirely outside of the optical axis of the patient's eye.

In another embodiment, a method for adjusting the refractive power of a fluid-filled intraocular lens implanted into a patient's eye is described. The method comprises ablating a portion on either one or both of an anterior region and/or a posterior region of the implanted fluid-filled intraocular lens. The ablating maintains the integrity of the fluid-filled intraocular lens.

In accordance with a first aspect, the ablated portion is on a surface of either one or both of the anterior and posterior portions.

In accordance with a second aspect, the ablated portion is disposed within a thickness of either one or both of the anterior and posterior portions. The ablated portion results in the creation of a hollow cavity.

### BRIEF DESCRIPTION OF THE DRAWINGS

Preferred and non-limiting embodiments of the inventions may be more readily understood by referring to the accompanying drawings in which:

FIGS. 1A and B are sectional views illustrating the certain anatomical features of the human eye with the lens in the unaccommodated and accommodated states, respectively.

FIGS. 2A, B and C are cut-away perspective, plan and cross-sectional views, respectively, of an embodiment of a fluid-filled IOL with circular ablation patterns.

FIGS. 3A and B are plan and side views, respectively, of an embodiment of a fluid-filled IOL with arcuate ablation patterns.

FIGS. 4A and B are comparisons of an astigmatic eye before and after ablation of the IOL, respectively.

FIGS. SA and B are plan and side views, respectively, of an embodiment of a fluid-filled IOL with an ablation-formed aperture.

FIGS. 6A and B are plan and side views, respectively, of another embodiment of a fluid-filled IOL with articulating flex regions.

FIGS. 7A and B are plan and side views, respectively, of another embodiment of a fluid-filled IOL with articulating flex regions and convex optical element.

FIG. 8 is a plan view, of another embodiment of a fluid-filled IOL with articulating flex regions.

FIG. 9 is a plan view, of a fluid-filled IOL with ablations within the thickness of the IOL's materials.

FIG. 10 is a flow chart demonstrating a method for post-operatively adjusting the refractive power of an IOL.

FIG. 11 is a flow chart demonstrating a method for post-operatively adjusting the amplitude of accommodation of an IOL.

FIGS. 12A and B are plan and side views, respectively, of an embodiment of a refractive optical element and haptic system.

FIGS. 13A, B and C are cut-away perspective, plan and cross-sectional views, respectively, of an embodiment of a refractive optical element and haptic system of FIGS. 3A-B coupled to a fluid filled lens capsule.

FIGS. 14A and B are plan and side views, respectively, of another embodiment of a refractive optical element and haptic system.

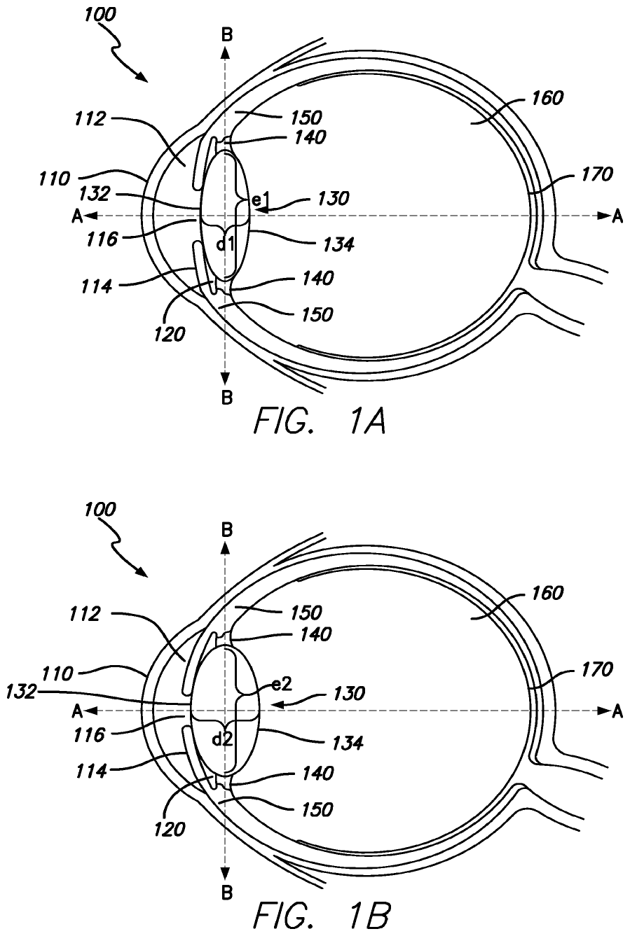

FIGS. 1A, B and C are cut-away perspective, plan and cross-sectional views, respectively, of another embodiment of a refractive optical element and haptic system of FIGS. 5A-B coupled to a fluid-filled lens capsule.

FIG. 16 depicts an embodiment of an intraocular lens device implanted in the posterior chamber of a human eye.

Like numerals refer to like parts throughout the several views of the drawings.

### DETAILED DESCRIPTION OF THE PREFERRED EMBODIMENTS

Specific, non-limiting embodiments of the present invention will now be described with reference to the drawings. It should be understood that such embodiments are by way of example only and merely illustrative of but a small number of embodiments within the scope of the present invention. Various changes and modifications obvious to one skilled in the art to which the present invention pertains are deemed to be within the spirit, scope and contemplation of the present invention as further defined in the appended claims.

As shown in FIGS. 1A-B, the human eye 100 comprises three chambers of fluid: the anterior chamber 112, the posterior chamber 120 and the vitreous chamber 160. The anterior chamber 112 corresponds generally to the space between the cornea 110 and the iris 114 and the posterior chamber 120 corresponds generally to the space bounded by the iris 114, the lens 130 and zonule fibers 140 connected to the periphery of the lens 130. The anterior chamber 112 and the posterior chamber 120 contain a fluid known as the aqueous humor, which flows therebetween through an opening that is defined by the iris 114, known as the pupil 116. Light enters the eye 100 through the pupil 116 and travels along a visual axis A-A, striking the retina 170 and thereby produce vision. The iris 114 regulates the amount of light entering the eye 100 by controlling the size of the pupil 116. Typically, in conditions of bright light, the pupil narrows to a diameter that is typically in the range of 3-5 mm and in conditions of darkness, the pupil may dilate to a diameter that is typically in the range of 4-9 mm.

The lens 130 is a clear, crystalline protein membrane-like structure that is quite elastic, a quality that keeps it under constant tension via the attached zonules 140 and ciliary muscles 150. As a result, the lens 130 naturally tends towards a rounder configuration, a shape it must assume for the eye 100 to focus at a near distance as shown in FIG. 1B. By changing shape, the lens functions to change the focus distance of the eye so that it may focus on objects at various distances, thus allowing a real image of the object of interest to be formed on the retina.

As shown in FIGS. 1A and 1B, the lens 130 may be characterized as a capsule having two surfaces: an anterior surface 132 and a posterior surface 134. The anterior surface 132 faces in an anterior direction towards the posterior chamber 120 and the posterior surface 134 faces a posterior direction towards the vitreous body 160. The posterior surface 134 contacts the vitreous body 160 in such a manner that fluid movements within the vitreous body 160 are communicated to the posterior surface 134 and may cause the shape of the lens 130 to change.

The eye's natural mechanism of accommodation is reflected by the changes in shape of the lens 130 which, in turn, changes the extent to which it refracts light.

FIG. 1A shows the eye 100 in a relatively unaccommodated state, as may be the case when the eye is focusing at a distance. In an unaccommodated state, the ciliary muscles 150 relax, thereby increasing the diameter of its opening and causing the zonules to be pulled away from the visual axis A-A. This, in turn, causes the zonules 140 to radially pull on the periphery of the lens 130 and cause the lens 130 to flatten. As the shape of the lens 130 is flattened, its ability to bend or refract light entering the pupil is reduced. Thus, in an unaccommodated state, the lens 130 has a flatter surface, its diameter e along the equatorial axis B-B is lengthened and its thickness d₁ along the visual axis A-A is decreased, all relative to the accommodated state (compare e₂ and d₂ in FIG. 1A).

FIG. 1B shows the eye 100 in a relatively accommodated state, as may be the case when the eye is focusing on a nearby object. In an accommodated state, the ciliary muscles 150 contract, and the contraction of the ciliary muscles 150 causes them to move in an anterior direction. This, in turn, reduces the stress on the zonules 140, thereby lessening the stress exerted by the zonules 140 on the lens 130. The lens 130 thereupon undergoes elastic recovery and rebounds to a more relaxed and accommodated state, in which the lens 130 has a more convex anterior surface, its diameter e₂ along the equatorial axis B-B is decreased and its thickness d₂ along the visual axis A-A is increased relative to the unaccommodated state (compare e₂ and d₁ in FIG. 1A). Although FIG. 1B depicts the anterior and posterior surfaces 132, 134 of the lens capsule 130 as having roughly the same radius of curvature, it is believed that during accommodation, the radius of curvature for the anterior surface 132 increases and the radius of curvature of the posterior surface 134 is not significantly changed from its unaccommodated state.

As demonstrated by FIGS. 1A and 1B, accommodation results from the changes in shape of the lens 130, including the changes in the thickness of the lens capsule 130 (d₁ vs. d₂), changes in the diameter of the lens capsule 130 (e vs. e₂) and the changes in the curvature of the anterior surface 132 of the lens capsule 130. While the ciliary muscles 150 are known to play a significant role in exerting these changes, it is believed that the vitreous body 160 also plays a significant role, primarily due to the nature of the contact between the posterior surface 134 of the lens 130 and the vitreous body 160, in which the posterior surface 134 responds to and transmits anterior fluid movement in the vitreous body 160 to effectuate changes in shape of the lens 130.

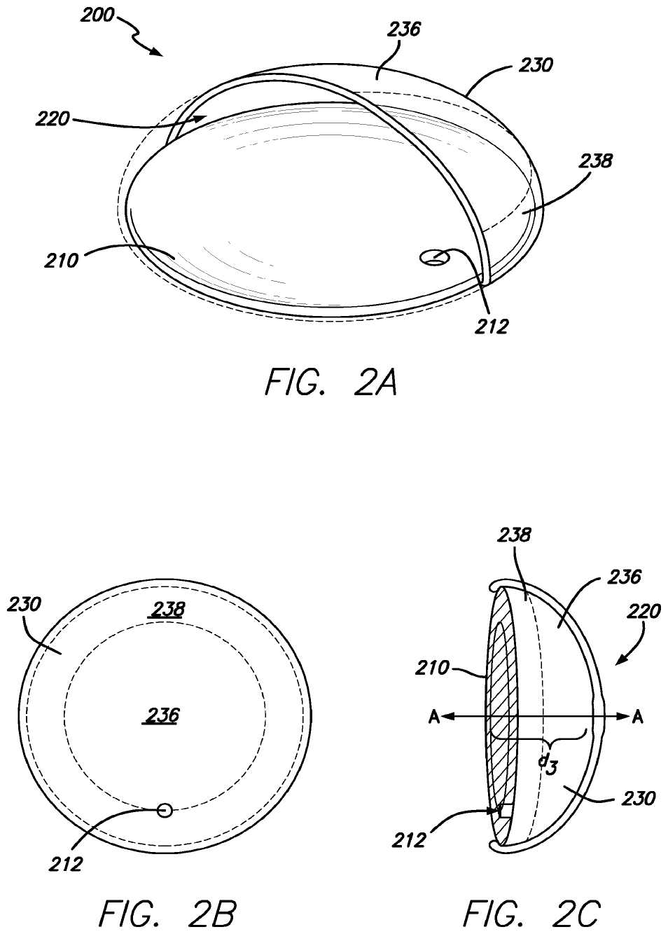

FIGS. 2A-2C illustrate an embodiment of an accommodating IOL device 200 that may be implanted into the lens capsule 130 of the eye following cataract removal. U.S. Patent Application Publication No. 2013/0053954 A1, filed on Oct. 26, 2012 and published on Feb. 28, 2013, discloses exemplary embodiments of accommodating IOL devices, the contents of which are incorporated herein by reference in its entirety as if fully set forth herein. The IOL device 200 is shown to comprise an optical element 210 and a flexible element 230 coupled to the optical element 210. The optical element 210 and the flexible element 230 together define an interior cavity 220 which may be filled with fluid. It is understood that the IOL device 200 may be implanted into the lens capsule 130 of the eye in at least one of two orientations. In a first orientation, the IOL device 200 may be implanted with the optical element 210 facing in an anterior direction and the flexible element 230 facing the posterior direction towards the vitreous body 160. In a second orientation, the IOL device 200 may be implanted with the optical element 210 facing in a posterior direction and the flexible element 230 facing in an anterior direction.

The optical element 210 may be made of plastic, silicone, acrylic, or a combination thereof. In accordance with a preferred embodiment, the optical element 210 is made of poly(methyl methacrylate) (PMMA), which is a transparent thermoplastic, sometimes called acrylic glass. The optical element 210 is preferably sufficiently flexible so as to change its curvature in response to the accommodating forces of the patient's eye.

In accordance with one embodiment, the optical element 210 is resiliently biased to a shape that approximates the shape of a natural and unaccommodated lens (see FIG. 1A). The optical element 210 accordingly increases its degree of curvature in response to the in response to the contraction of the ciliary muscles and is resiliently biased to a flatter configuration or a decreased degree of curvature in response to the relaxation of the ciliary muscles.

In accordance with another embodiment, the optical element 210 is resiliently biased to a shape that approximates the shape of a natural and accommodated lens (see FIG. 1B). The optical element 210 accordingly is resiliently biased to a convex configuration similar to that of the natural lens in the accommodated state and assumes a less convex configuration as the ciliary muscles 150 relax and the tension of the zonules 140 on the lens capsule 130 increases.

In engaging the zonules 140, the IOL device responds to part of the accommodative mechanism of the eye in which the ciliary muscles 150 and the zonules 140 cause a bilateral movement of the optical element 210 along the optical axis to thereby provide part of the accommodating response.

The optical element 210 is preferably sufficiently flexible so as to change its curvature in response to the contraction/relaxation of the ciliary muscles. In a preferred embodiment, the optical element 210 is resiliently biased to a shape that approximates the shape of a natural and unaccommodated lens (see FIG. 1A). The optical element 210 accordingly increases its degree of curvature in response to the anterior force exerted by the vitreous body and is resiliently biased to a flatter configuration or a decreased degree of curvature, similar to the configuration of the natural lens in the unaccommodated state, in the absence of the anterior force.

The flexible element 230 may be constructed from any biocompatible elastomeric material. In a preferred embodiment, the flexible element 230 has an external surface that approximates the posterior surface of the lens capsule adjacent the vitreous body. The flexible element 230 is preferably configured and shaped to contact a substantial, if not the entire, area of the posterior surface of the lens capsule. In a particularly preferred embodiment, this point of contact is at and around the optical axis of the posterior surface.

In accordance with another preferred embodiment, the IOL device 200 may be configured to resiliently assume a shape having a width d3 that is substantially equal to the width of the lens capsule 130 accommodated eye (see d2 of FIG. 1B) when it is implanted in the patient's eye. This may be achieved by constructing the IOL device 200 with resilient materials having some degree of shape memory and also by filling the cavity 220 with a volume of fluid sufficient to expand the flexible element 230 to the desired width, d3.

The flexible element 230 may preferably be made from a polyvinylidene fluoride (PDVF) material.

Once the IOL device is implanted in the lens capsule of the patient, a volume of fluid may be injected into the cavity 220 via an injection port 212. In one preferred embodiment, the fluid may be an aqueous solution of saline or hyaluronic acid and does not provide a significant, or any, contribution to the refractive power of the 10C device. In another preferred embodiment, the fluid may have a viscosity that is substantially the same as the vitreous humor. In yet another preferred embodiment, the fluid may have a refractive index that is substantially the same as the aqueous humor or the vitreous humor. In a particularly preferred embodiment, the fluid may be a polyphenyl ether (PPE). PPE provides twice the refractive index as water and is described in U.S. Pat. No. 7,256,943, issued Aug. 14, 2007, the entire contents of which are incorporated by reference as if fully set forth herein.

The precise volume of fluid injected into the cavity 220 may differ based on the subject's anatomy, among other factors. The volume of fluid injected into the cavity 220 is not critical so long as it is sufficient to expand the flexible element 230 such that the posterior portion of the flexible element 230 substantially contacts the posterior portion of the lens capsule and engages the vitreous body of the subject's eye. As explained above, in one preferred embodiment, a volume of fluid is injected into the cavity 220 so as to provide a width d3 of the IOL device along the optical axis A-A substantially approximating the lens width d2 of the accommodated eye 100. In another preferred embodiment, a volume of fluid is injected into the cavity 220 so as to provide a width d3 of the IOL device along the optical axis A-A substantially approximating the width d₁ of the unaccommodated eye 100.

Once the IOL device is implanted in the lens capsule of the patient, it may be desirable to make adjustments to the refractive characteristics of the IOL device or to change its ability to respond (i.e., change curvature) to the contraction/retraction of the ciliary muscles. Post-implantation changes are particularly desired to optimize the vision correction or range of accommodation of the already-implanted IOL device. It is often difficult to predict, with absolutely precision, the refractive characteristics or the amplitude of accommodation that will be required before implantation. Errors may arise from errors in geometrical measurements of the eye, post-surgical changes in the lens position, unexpected anatomical features of an eye, etc.

In accordance with one preferred embodiment, an energy source may be used to ablate at least a portion of a surface of the IOL, in situ and post-surgically, to modify the characteristics of the IOL. For example, the geometry of the IOL (e.g., shape and/or curvature) may be modified so as to effectuate a change in the refractive power (sphere, cylinder, and axis) to a desired value. The characteristics of the IOL are modified in such a way that it further responds to normal ciliary and zonular forces in the eye to achieve either larger or smaller amplitude of accommodation.

The energy source used to perform the ablation may include a laser, radio-frequency (RF) energy, microwaves, or X-rays. Inductive heating and chemical reactions may also be used to alter the refractive characteristics of the IOL. For example, inductive heating may be used by embedding materials within the IOL, wherein the embedded materials alter the characteristics of the IOL by heating up when exposed to a magnetic field. Similarly, materials may be embedded in the IOL that react to specific wavelengths of energy such that, when exposed to these wavelengths, a change is effectuated in the refractive characteristics of the IOL.

Several examples of ablating the IOL will be discussed with respect to the following figures. Ablation is understood to include, but not require, removal of material by erosion, melting, vaporization. Accordingly, ablation may also include a remodeling or reshaping of material without the removal of material through application of an energy source. As described herein, ablation patterns may be made to either one or both of the optical element 210 and/or the flexible element 230 to effectuate changes in the amplitude of accommodation and refractive characteristics of the IOL. Even where ablation is discussed only with respect to the flexible element 230, it is understood that the ablation may be performed on either one or both of the opposing sides of the fluid filled IOL device after implantation.

The ablation may be performed on the surfaces of the IOL that faces anteriorly, posteriorly or both to achieve the desired result. Where the ablation is performed on both anterior and posterior surfaces of the implanted IOL lens, a significantly large change in the amplitude of accommodation or refractive power may be observed.

Alternatively, rather than ablating an inner or outer surface of the IOL, ablation may also be performed within the IOL materials, as will be further explained below. It is further understood that the implanted IOL device 200 may be implanted in the lens capsule of the eye in such a manner that the refractive or optical element 210 may be positioned in either one of the anterior or posterior direction and the flexible element 230 may be positioned in the other one of the anterior or posterior direction, both along an optical axis. The figures and explanations are provided by way of example only, and the present invention is not limited to these examples.

In one embodiment, a laser may be used to ablate the surface of the optical element 210 and/or the flexible element 230 to provide a thinner surface. For example, in FIGS. 2A-2C, the entire surface of either one or both of the optical element 210 and the flexible element 230 may be ablated by a laser, resulting in a thinner surface. The modified, thinner surface of the flexible element 230 would produce a greater amplitude of accommodation in response to the contraction and relaxation of the ciliary muscles.

The phrase “amplitude of accommodation” is understood to mean the degree of change in curvature of the IOL in response to the contraction and relaxation of the ciliary muscles.

As was described above with respect to FIG. 1, when the ciliary muscles 150 relax, they increase the diameter of its opening and cause the zonules 140 to be pulled away from the visual axis A-A, which results in the zonules pulling the IOL to a flattened, unaccommodated state. The natural resistance of the IOL materials, including the flexible element 230 and the optical element 210, are partially responsible for determining how much force is required to flatten the IOL to an unaccommodated state. And, in the opposite situation, when the ciliary muscles 150 contract, they decrease the diameter of the opening, causing the zonules 140 to move inwards, and IOL to become more curved (the “accommodated” state of FIG. 1B).

The now-thinner ablated surface of the IOL, whether it is the flexible element 230, the optical element 210, or both, would yield a greater change in curvature in response to the forces exerted by the ciliary muscles and the zonules because the thinner surfaces naturally provide less resistance to these forces. When the ciliary muscles contract, the now-thinner material of the flexible element 230 would provide less resistance, thereby yielding greater changes in curvature in response to the contraction of the ciliary muscles. The greater amplitude of accommodation created in either one or both of the optical element 210 and the flexible element 230 provides a change in curvature of the IOL and a change in the refractive power of the IOL.

Alternatively, rather than ablating an entire surface, portions of the optical element 210 and/or the flexible element 230 may be selectively ablated to create the desired effect on amplitude of accommodation and refractive power. For example, in FIGS. 2A-2C, an interior region 236 and an exterior region 238 may be defined on either one or both of the optical element 210 and/or the flexible element 230. There may be more than two regions, and the shapes and sizes of the regions may be varied according to the desired effect. In the embodiment shown in FIGS. 2A-2C, the interior region 236 around the optical axis A-A may be selectively ablated so as to thin only the interior region 236, while leaving exterior region 238 at its original thickness. This would result in an increased amplitude of accommodation for the IOL device 200.

Conversely, a circumferential region 236 surrounding the interior region 236 may be selectively ablated and thinned. This would result in generally decreasing the curvature of the IOL about its optical axis A-A. While FIGS. 2A-C depict the interior region 236 and the exterior region 238 as being disposed on the flexible element 230, it is understood that these regions may be similarly disposed on the optical element 210 (a) in place of being disposed on the flexible element 230 to produce similar results with respect to the amplitude of accommodation or (b) in addition to being disposed on the flexible element 230 to provide an increased or decreased amplitude in accommodation in a bilateral direction.

In accordance with one embodiment, the diameter of the circumferential region 238 ablated is based on the size of a pupil, whether it is completely dilated or contracted. The average diameter of a pupil is about 3-5 mm in light conditions and 4-9 mm in dark conditions. Thus, the average diameter of the circumferential region 236 may range anywhere from about 3 mm to 9 mm, depending on the desired effect on accommodation and vision.

In addition to providing a range of accommodation, the IOL device may be used to treat various ophthalmic conditions. For example, bene dilitatism is a condition that is typified by chronically widened pupils due to the decreased ability of the optic nerves to respond to light. In normal lighting, people afflicted with this condition normally have dilated pupils, and bright lighting can cause pain. Thus, in one embodiment, the circumferential region 236 may be ablated to control the light entering the pupil for those suffering from this condition.

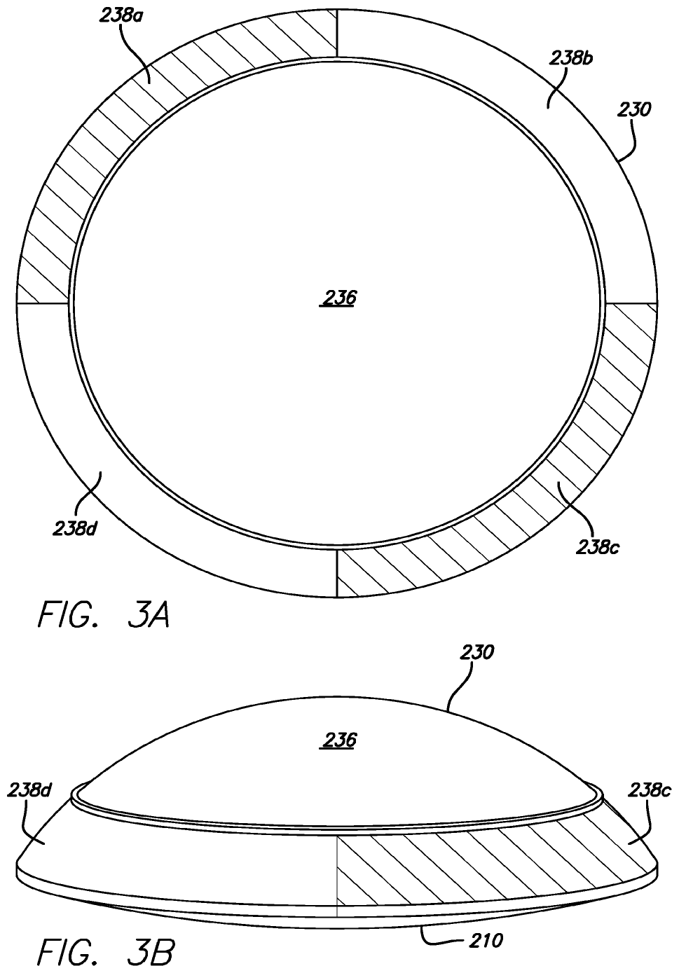

The ablation patterns may be symmetric or asymmetric. Asymmetric modifications may also be made so as to alter the shape of the IOL. Such asymmetric modifications may be useful in correcting astigmatisms, which are the result of an irregularly shaped lens. In one embodiment, asymmetric modifications may be made to the IOL by ablating certain arc segments of the circular regions 236, 238, as shown in FIGS. 3A and 3B. The exterior region 238 has been divided into four 90-degree arc lengths, 238a, 238b, 238c, and 238d. By ablating only certain arc-segments, the IOL may be modified to flex into non-circular curvatures. In FIGS. 3A and 3B, only arc lengths 238a and 238c have been ablated. In a further embodiment, when used to treat symmetric astigmatisms, the ablated “arc lengths” may be pair-matched so that if a particular arc length is ablated, the corresponding arc length 180 degrees across from the ablated arc length is also ablated. Arc lengths 238a and 238c are two such “pair-matched” arc lengths. While four arc lengths are shown here, more, or fewer, arc lengths may be used. Where arc lengths are to be “pair-matched,” the circular regions may be divided into an even number of arc length segments so as to create a whole number of arc length pairs.

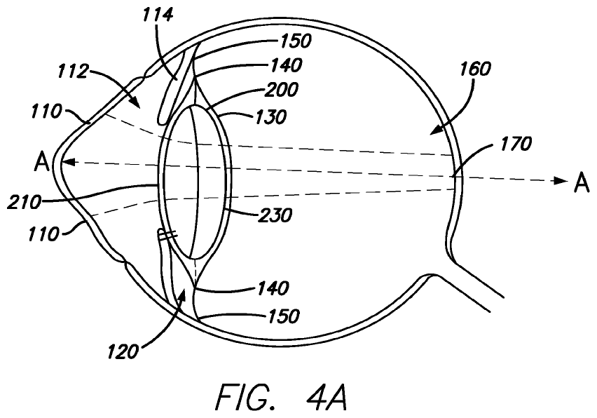

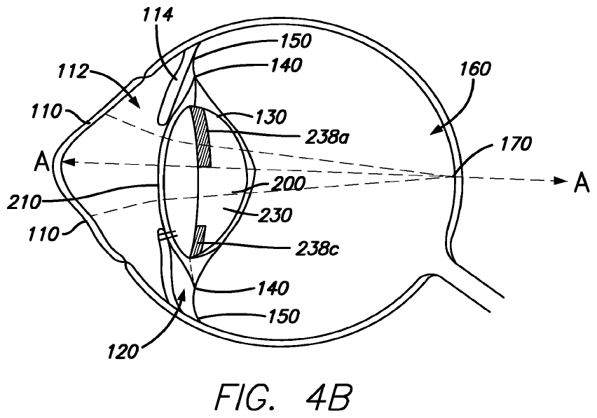

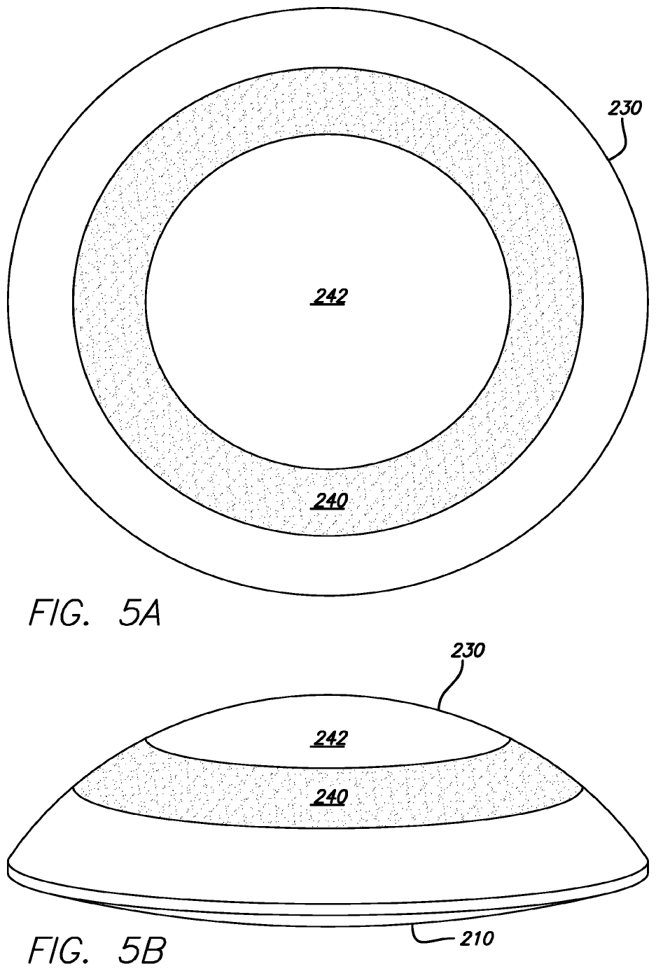

Astigmatism occurs when the cornea is misshapen. The misshapen cornea causes images to be distorted or elongated because the light entering the eye is not correctly focused on the retina. This is depicted in FIG. 4A, where it can be seen that light enters the astygmatic cornea 110 and passes through the IOL 200, but does not come to a focus at the retina 170. The shape of the IOL must be changed so as to compensate for the misshapen cornea, and properly focus the light. The shape of the IOL may be altered by selectively ablating the IOL, as was described above. In FIG. 48, the same astigmatic cornea 110 is shown with an IOL 200 that has been selectively ablated to alter its shape. The IOL 200 has been ablated and thinned out at arcuate sections 238a and 238c. These ablations have resulted in the rear portion of the IOL 200 being curved in such a way that the path of the light entering the eye is altered so that it now comes to a focus at the retina 170.

In yet a further embodiment, the IOL may be ablated so as to create and/or alter an aperture in the IOL, as demonstrated in FIGS. 5A and 5B. A ring-shaped area 240 has been ablated to create an aperture 242. The aperture 242 may be an unobstructed area through which light may pass through substantially unimpeded, rather than an actual opening or hole within the IOL. The creation of the aperture 242 may be customized to each patient to optimize centration and tilt of the IOL in regards to the pupil and/or the optical axis. The size of the aperture 242 may be modified to alter the depth of field, wherein a smaller aperture creates a large depth of field.

In one embodiment, this aperture 242 may be created by ablating a ring 240 around the optical axis to scatter incoming light. For example, the ring 240 may be created by ablating the area to create a rough surface which results in 85 to 95% of incoming light being scattered. Thus, only a small area in the center of the IOL, the aperture 242, would allow for focused light to pass through. Rather than ablating a rough surface to scatter light, the light-scattering ring 240 may be created using a color-changing material placed within the IOL that changes to a darker, light-blocking color when ablated with lasers. Examples might include e-paper and polarization paper.

While a smaller aperture 242 will grant the patient a greater depth of field, there is a trade-off between depth of field and contrast. As the aperture 242 gets smaller, depth of field is increased, but contrast is decreased. Therefore, the diameter of the aperture 242 may be chosen such that the depth of field is increased while not sacrificing too much contrast. The circumferential ablation pattern is defined as having an inner diameter which defines a non-ablated central portion and an outer diameter which includes both the non-ablated and ablated portions. In a preferred embodiment, the inner diameter is in the range of 1 mm to 2 mm, preferably 1.2 mm to 1.8 mm and most preferably about 1.5 mm to 1.7 mm. In accordance with the most preferable embodiment, the inner diameter is about 1.6 mm. In an alternative embodiment, the size of the aperture 242 may be selected by dilating the pupils of the patient and measuring the size of the patients pupils when they are dilated and undilated. These measurements may then be used to determine what the appropriate size of the aperture 242 is.

In a preferred embodiment, the ring 240 ablated around the aperture 242 is substantially, if not completely, opaque and the amount of light entering the retina is determined by the size or diameter of the aperture 242.

Additionally, smaller apertures result in an overall decrease in brightness observed by the patient. In a preferred embodiment, an aperture may be created in only one of the patient's eyes so that the patents depth of field is increased, but observed brightness is not decreased to an uncomfortable degree.

Post-surgical ablations to the IOL may be performed by a laser, preferably using either one of a YAG or femtosecond laser. Femtosecond lasers typically achieve precise ablation of tissues with high resolution without causing significant damage to the surrounding tissues. Femtosecond lasers also have the ability to ablate polymer materials with the same precision and resolution and hence are suitable for effecting precise geometrical changes in the implanted IOLs after their implantation and settlement in the eye. These lasers have the ability to focus their energy such that even the thinnest lens and/or membranes may be ablated in a controlled and precise manner. Most such ablations are performed so that the optical zone of the eye has no significant interferences.

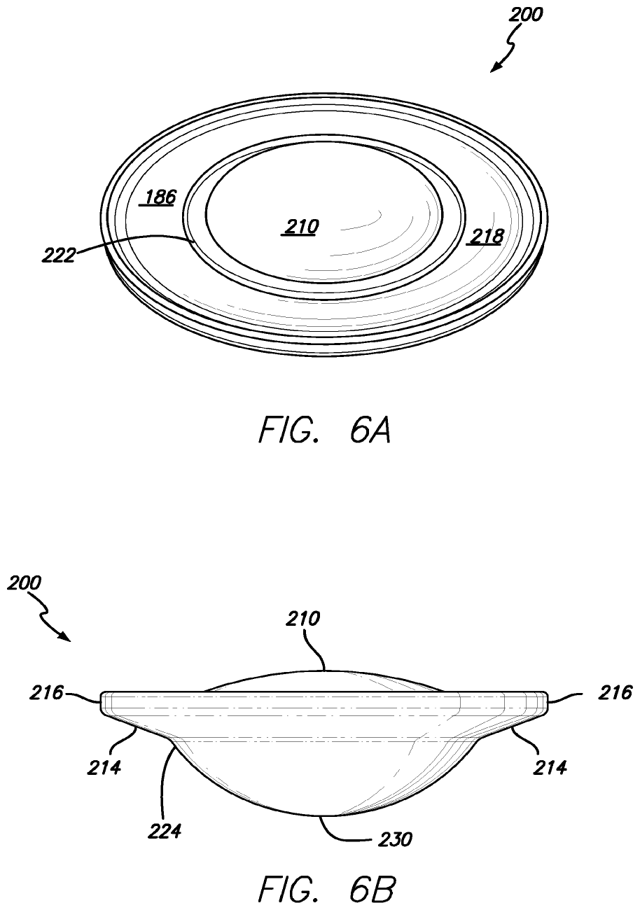

FIGS. 6A-B illustrate another embodiment of an accommodating IOL device 200 that may be implanted into the lens capsule 130 of the eye following cataract removal. U.S. patent application Ser. No. 13/725,895, filed on Dec. 21, 2012, discloses several embodiments of accommodating IOL devices, the contents of which are incorporated herein by reference in its entirety as if fully set forth herein. The IOL device 200 is shown to comprise an optical element 210, a flexible element 230, and an articulating member coupling the lens 210 and surface 230 together. The articulating member is depicted as comprising an anterior member 218, a posterior member 214, and a peripheral portion 216 therebetween. In a preferred embodiment, the peripheral portion 216 defines the circumference of the IOL device 200. The accommodating IOL device 200 is depicted as heaving a biconvex exterior surface when the enclosed cavity 220 is filled with a fluid. The IOL device 200 further has a plurality of flex regions or hinges to permit the optical element 210 and the flexible element 230 to reciprocate away and towards one another along an optical axis A-A. The inclusion of an anterior flex region 222 between the optical element 210 and the anterior member 218 and a posterior flex region or hinge 224 between the flexible element 230 and the posterior member 214 permit a greater degree of displacement along the optical axis in opposing directions when opposing sides of the peripheral portion 216 move towards one another. Both the anterior member 218 and the posterior member 214 are angled away from one another so as to facilitate a reciprocal displacement away from and toward one another in response to the accommodating forces of the eye.

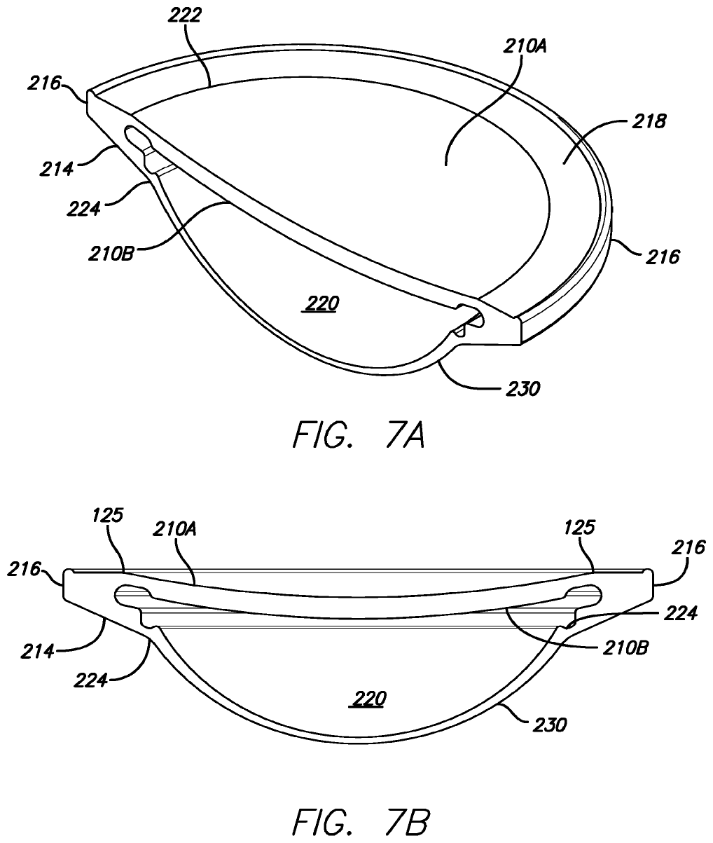

FIGS. 7A-B depict a slightly different embodiment of the IOL 200. While FIGS. 2-5 depict the optical element 210 as being biconvex, it should be understood that the optical element 210 may be biconcave or have a combination of a convex or concave outer surface and a concave or convex inner surface. FIGS. 7A-B depict the optical element 210 as having an outer concave surface 210A and an inner convex surface 210B. In this embodiment, the presence of anterior hinges 222 is optional since both the optical element 210 and the flexible element 230 are displaced in the same posterior direction when opposing sides of the peripheral portion 216 move towards one another.

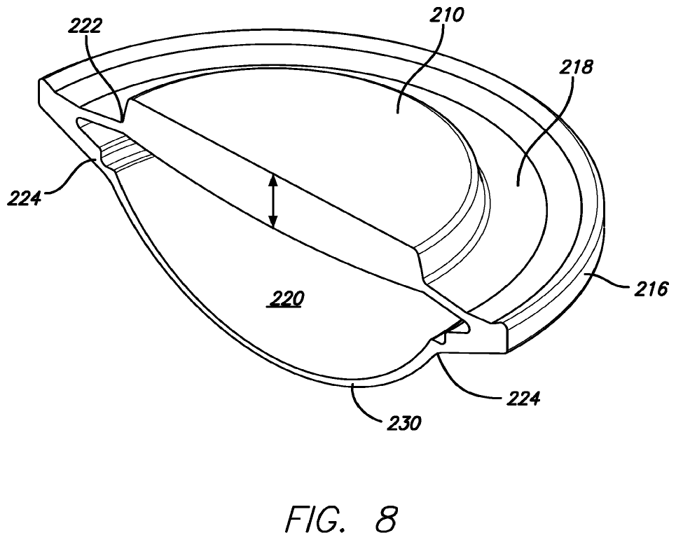

FIG. 8 depicts yet another embodiment in which the IOL device comprises an optical element 210 having a thickness that protrudes in the anterior direction. In the embodiment depicted in FIG. 8, the optical element 210 and the flexible element 230 move away from one another when opposing sides of the peripheral portion 216 are displaced towards one another. The thickness of the optical element 210 may be ablated using a laser to alter the refractive properties of the IOL, as was discussed above with respect to the previous embodiments.

In the embodiments shown in FIGS. 6-8, the degree of accommodation is influenced in part by the movement of flex regions or hinges 222 and 224. As such, in addition to or instead of ablating the optical element 210 and/or the flexible element 230, the flex regions 222, 224 may be ablated to alter the amplitude of accommodation of the IOL 200. These additional flex regions 222, 224 provide alternative methods of altering the amplitude of accommodation of the IOL and, as such, may provide greater control over how the IOL is modified.

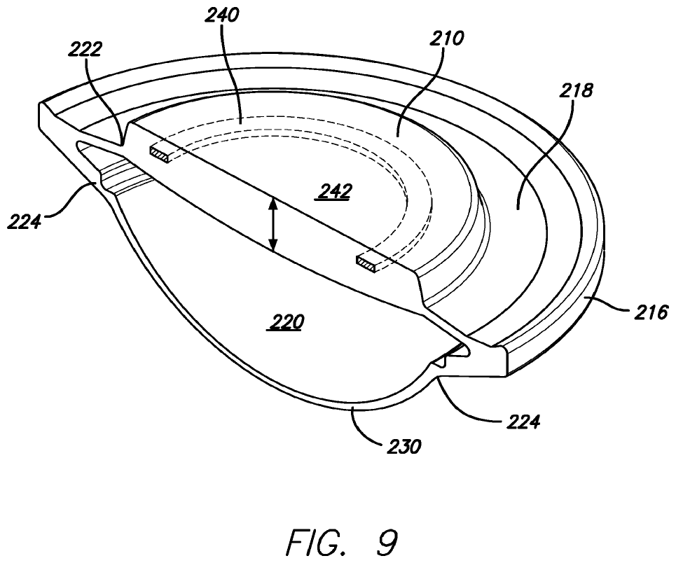

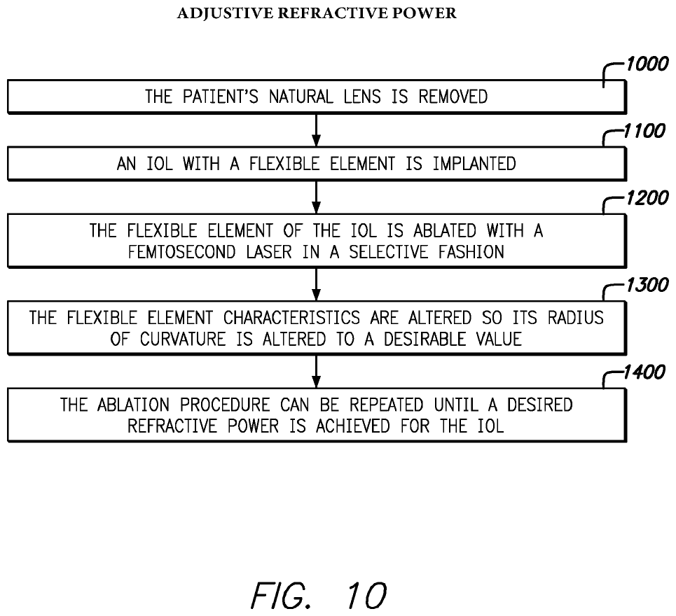

Rather than ablating an inner or outer surface of the IOL, the refractive characteristics of the IOL may also be altered by ablating within the thickness of the IOL material. An example of such an ablation is shown in FIG. 9. In FIG. 9, an area within the thickness of the IOL is ablated to create a void. This area may be ablated in a ring 240 and may define an unablated central portion 242. Thus, in contrast to FIG. 5, rather than ablating an outer surface of the IOL, the ring 242 is ablated within the thickness of the optical element 210. Ablating within the thickness of a material may be preferable in some instances because it may have less impact on the optical clarity of the IOL than surface ablations.

In another embodiment, the step of ablating within the thickness of a material may be performed by embedding materials within the IOL such that when those materials are exposed to a specific energy source, possibly identified by wavelength, the materials react to effectuate structural or chemical changes within the material or to vaporize or remove the materials.

For example, inductive heating may be used such that when embedded materials are exposed to magnetic fields, they heat up and cause ablation of material within the thickness of the IOL. Alternatively, laser energy may be used to ablate within the thickness of the IOL's materials by causing the lasers energy to focus on a point within the thickness of the material such that areas outside of the lasers focal point would not be ablated.

When using a laser or other energy source to ablate within the thickness of a material, the diameter of the lasers ablation sphere, a.k.a. the “laser spot size” may be adjusted according to the thickness of the material being ablated. For example, the thickness of the optical element 210 may be ˜1 mm thick, whereas the thickness of the flexible element 230 may be ˜100 microns in thickness. As such, whereas a laser spot size of ˜0.25 mm may be appropriate for ablating within the thickness of the optical element 210, the same laser spot size would ablate through the entire surface of the flexible element 230 if focused within its thickness.

Thus, where “internal” ablations are performed within the thickness of the material, the laser spot size is less than about 50% of the thickness of the material being ablated, preferably less than 25% of the thickness of the material, and even more preferably, less than 10% of the thickness of the material being ablated.

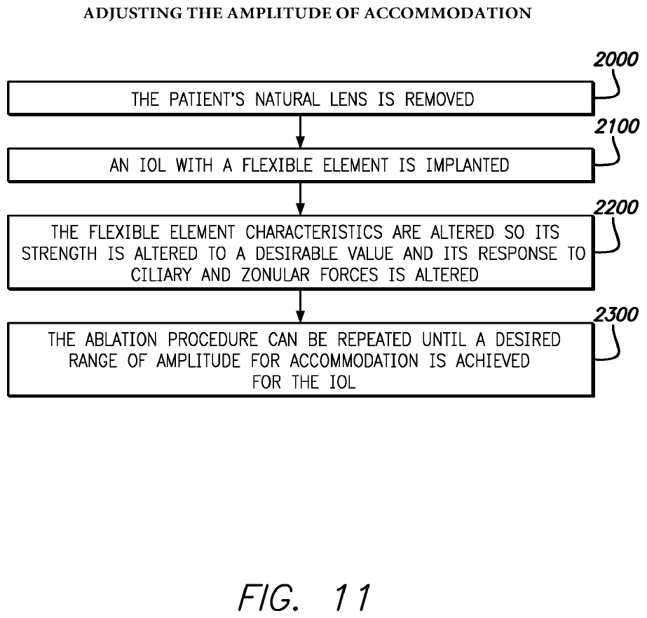

FIG. 10 outlines a process for replacing a patients natural lens with an IOL lens which is post-operatively ablated with a laser to change its radius of curvature to a desired value, as was discussed in greater detail above. In step 1000, the patients natural lens is removed. In step 1100, an intraocular lens with a flexible element is implanted. In step 1200, the flexible element is ablated with a femtosecond laser in a selective fashion. In step 1300, the flexible element characteristics are altered so that its radius of curvature is altered to a desirable value. In step 1400, the ablation procedure is repeated until a desired refractive power is achieved for the IOL.

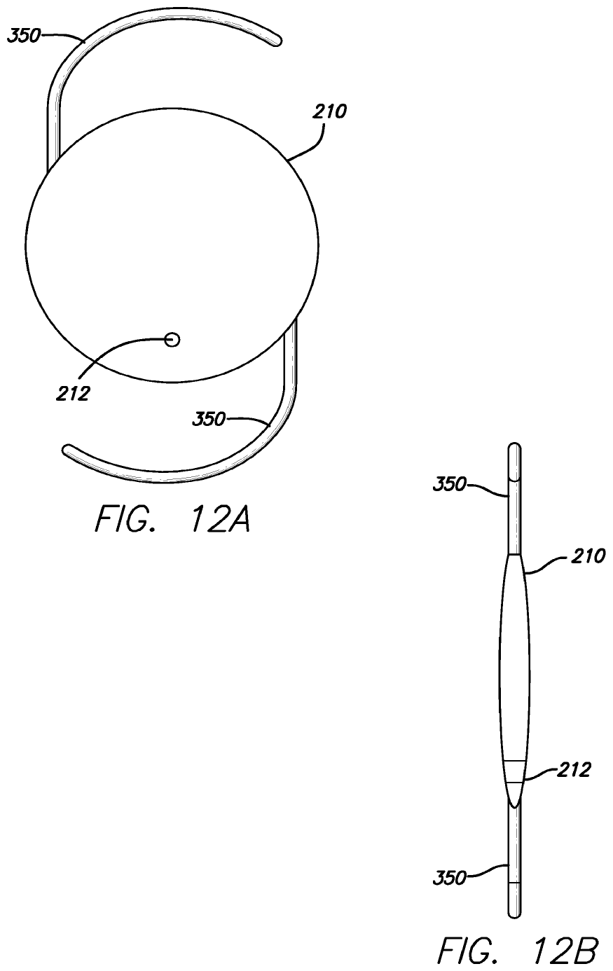

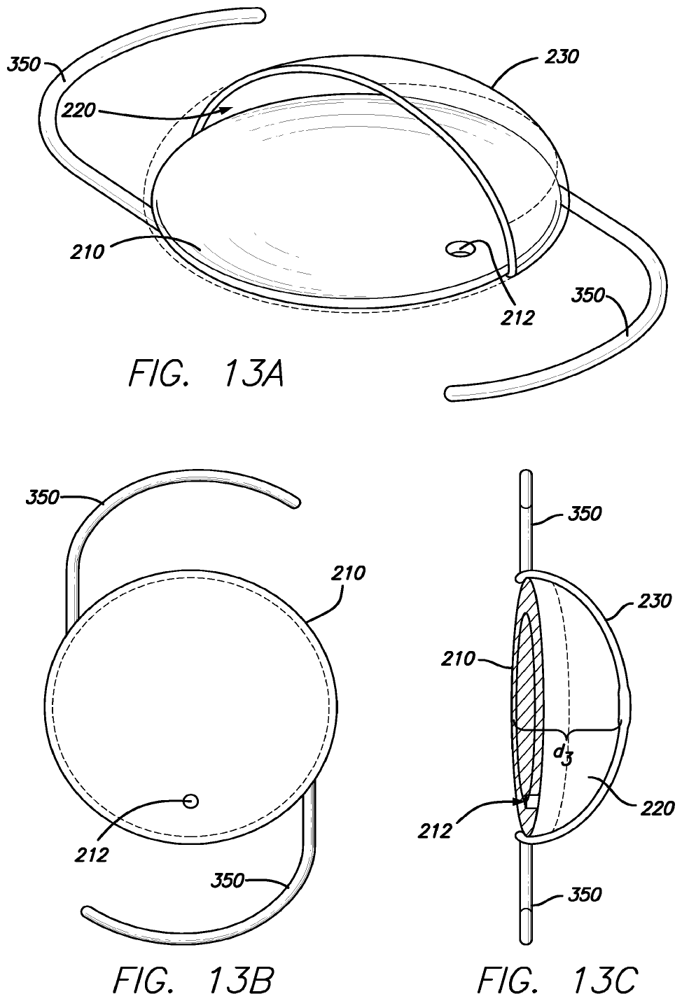

FIG. 11 outlines a process for replacing a patients natural lens with an IOL lens which is post-operatively ablated with a laser to change its strength to a desired value so that its refractive power in response to the eye's natural accommodative process is altered. In step 2000, the patient's natural lens is removed. In step 2100, an IOL with a flexible element is implanted into the patient's eye. In step 2200, the flexible elements characteristics are altered through ablation so that its strength is altered to a desirable value and its response to ciliary and zonular forces is altered. In step 2300, the ablation procedure is repeated until a desired range of amplitude for accommodation is achieved for the IOL.

A haptic system may be incorporated with the IOL device to position the optical element 210 at the optical axis A-A when implanted in the subjects eye. As it is preferable to center the optical element 210 relative to the optical axis A-A, the haptic system preferably comprises a plurality of haptic members extending radially from the IOL device and engaging the zonules 140 surrounding the lens capsule 130 of the eye.

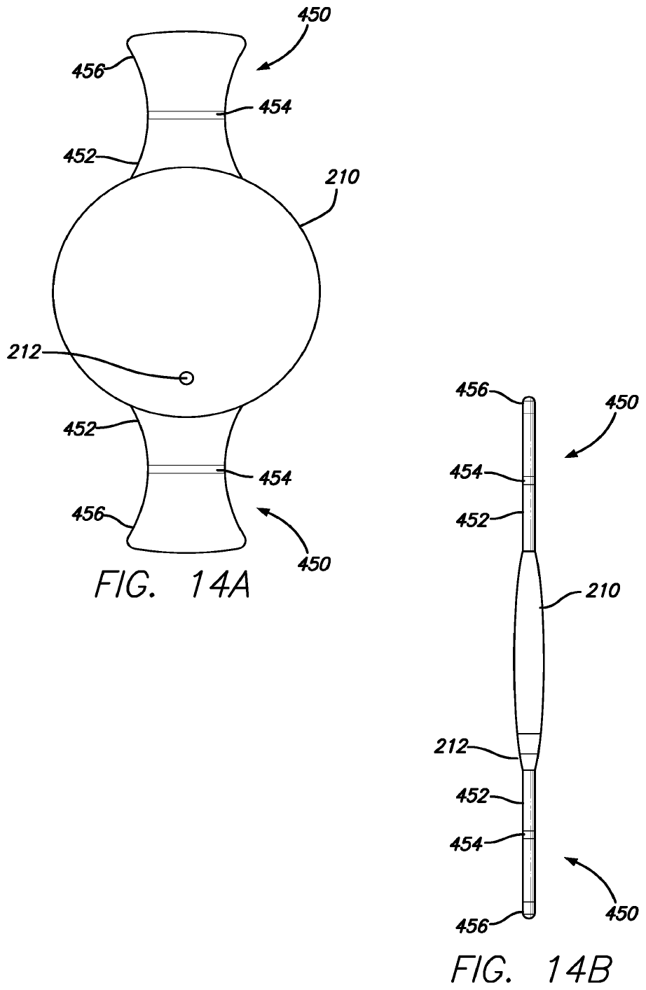

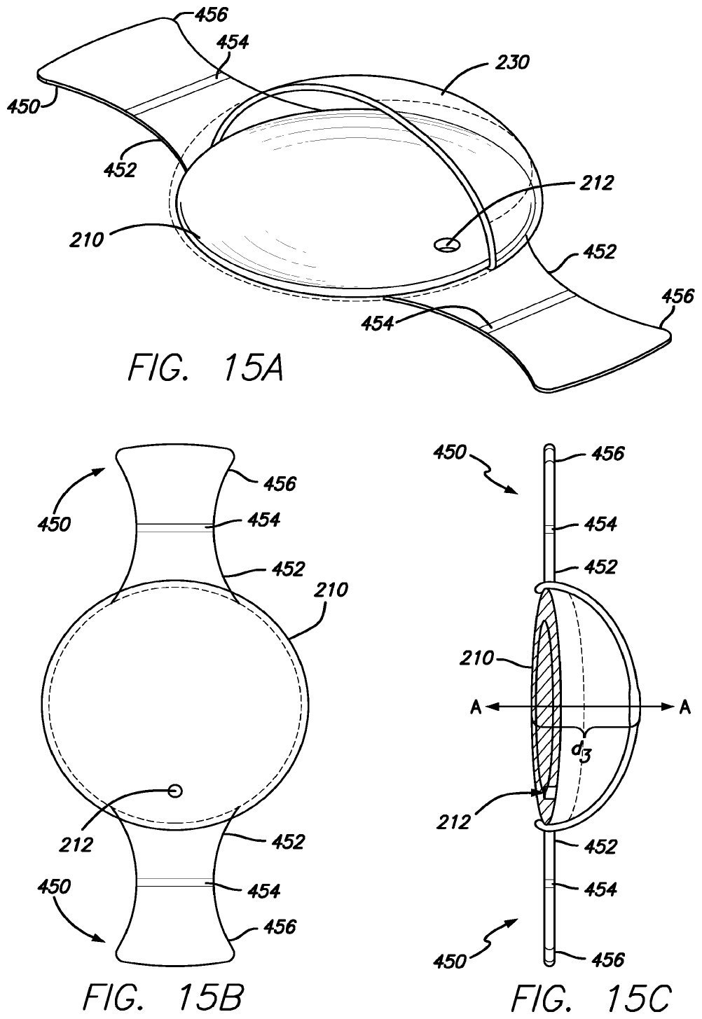

FIGS. 12A-12B depict an optical element 210 comprising a pair of spring haptics 350 coupled to opposing sides of the optical element 210. As further shown in FIGS. 13A-13C, a flexible element 230 may be coupled to the optical element 210/haptic 350 assembly along the periphery of the optical element 210. A seal is effectuated between the flexible element 230 and the periphery of the optical element 210 by laser welding and any other means known to those of skill in the art.

In another embodiment, the optical element 210 may be contained within a flexible element 230 that fully encloses the optical element 210. In accordance with this element, the flexible element 230 has a bag or balloon-like configuration and the spring haptics 350 may be attached either (1) to the optical element 210 itself and protrude from a sealed opening in the flexible element 230 or (2) to the flexible element 230. Although FIGS. 12-13 depict a pair of spring haptics 350 extending radially from the optical element 210, it is understood that any number of spring haptics 350 may be provided so long as optical element 210 is centered about the optical axis A-A when the IOL device is implanted in the eye.

In addition to changing the refractive characteristics of the IOL by changing the amplitude of accommodation and curvature characteristics of the IOL, the present disclosure may also be used to change the refractive characteristics of the IOL by displacing the IOL axially along the optical axis in either one of the anterior or posterior direction. The position of the IOL may be changed by ablating the haptic system described in FIGS. 11-12. The effective bending modulus of the haptic may be altered by ablating and thinning the spring haptics 350 at selective locations so as to achieve the desired result. This would result in differing forces applied by the spring haptics 350 in positioning the IOL, resulting in changes to the IOL's position, thereby resulting in changes to the IOL's refractive properties.

In one embodiment, a groove may be ablated across an anterior or posterior surface of the haptic to bias the IOL device in the posterior or anterior directly, respectively, along the optical axis A-A. In another embodiment, grooves may be ablated on both sides of the haptic to make the haptic generally less rigid and more amenable to actuating the IOL device in either the posterior or anterior direction in response to the accommodating forces. Ablated grooves may go entirely across the surface of the haptic, or partially across the surface of the haptic, depending on the desire result.

FIGS. 12A-B depict an alternative haptic system, with optical element 210 comprising a pair of plate haptics 450 coupled to opposing sides of the optical element 210. The plate haptics 450 comprise a pair of plate members each comprising a first end 452 attached to the optical element 210 and a second end 456 configured to engage the zonules 140 of the eye 100 when implanted in the lens capsule 130. A hinge 454 is disposed between the first and second ends 452, 456, to allow lateral movement of the optical element 210 in the anterior and posterior directions as the ciliary muscles 150 relax and contract, respectively. As further shown in FIGS. 13A-C, a flexible element 230 may be coupled to the optical element 210/haptic 450 assembly along the periphery of the optical element 210.

In another embodiment, the optical element 210 may be contained within a flexible element 230 that fully encloses the optical element 210. In accordance with this element, the spring haptics 350 may be attached either (1) to the optical element 210 itself and protrude from a sealed opening in the flexible element 230 or (2) to the flexible element 230. Although FIGS. 12-13 depict a pair of plate haptics 450 extending radially from the optical element 210, it is understood that any number of plate haptics 450 may be provided so long as optical element 210 is centered about the optical axis A-A when the IOL device is implanted in the eye.

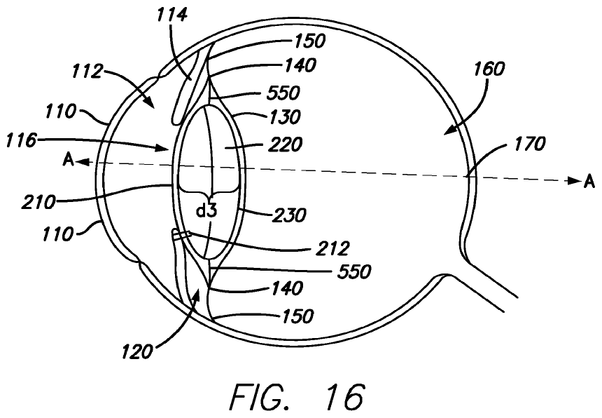

Similar to what was discussed with respect to the spring haptics in FIGS. 12-13, the plate haptics in FIGS. 14-15 may be ablated so as to alter their positioning characteristics. The characteristics of the plate haptics 450 may be altered by ablating the hinge 454. Alterations to the hinge 454 would result in a change in the haptics' positioning characteristics as the ciliary muscles 140 relax and contract. The resulting change in the optical element 210's positioning would yield a change in the refractive characteristics of the IOL. Also, similarly to the spring haptics 350, the plate haptics 450 may be ablated on one side or both the anterior and posterior sides so as to affect the positioning of the IOL.

FIG. 16 depicts an embodiment of the accommodating IOL device implanted in the lens capsule 130 of the eye in an accommodated state. Because both the optical element 210 and the flexible element 230 of the IOL device 200 is sufficiently flexible, it may be folded or rolled compactly prior to implantation, thereby requiring only a small incision of a few millimeters for insertion into the eye. As shown in FIG. 16, after the IOL device is implanted and the cavity 220 is filled with fluid, the IOL device is divided roughly in two: the anterior lens portion 210 facing the posterior capsule 120 and the flexible element 230 facing the vitreous body 160. The width d3 of the IOL device is resiliently biased to having a width that is roughly equal to the width of the natural lens capsule when it is in an accommodated state (see d2 of FIG. 1B). The flexible element 230 has an area of contact that approximates the surface area of the posterior portion 134 of the lens capsule 130 (See FIGS. 1A-B). Two or more haptics 550 are shown to protrude from the IOL device to substantially center the anterior lens portion 210 along the optical axis A-A.

The accommodated IOL device shown in FIG. 16 is implanted in the lens capsule of a subject's eye by introducing an IOL device in the lens capsule of the subject's eye through a small incision in the subject's eye, wherein the IOL device comprises a refractive optical element 210 coupled to a flexible element 230 to define an internal cavity 220. The IOL device is then positioned within the lens capsule 130 of the subject's eye to substantially center the refractive optical element 210 along an optical axis A-A. A volume of fluid is then injected into the internal cavity 220 of the IOL device sufficient to cause the flexible element 230 to contact the posterior portion of the lens capsule which, in turn, contacts the vitreous body in at least an area at and surrounding the optical axis A-A. In a preferred embodiment, the volume of fluid injected into the internal cavity 220 is sufficient to produce a width d3 of the IOL device along the optical axis A-A that is substantially equal to the width of a natural lens capsule in an accommodated state.

The invention described and claimed herein is not to be limited in scope by the specific preferred embodiments disclosed herein, as these embodiments are intended as illustrations of several aspects of the invention. Indeed, various modifications of the invention in addition to those shown and described herein will become apparent to those skilled in the art from the foregoing description. Such modifications are also intended to fall within the scope of the appended claims.

1. A method for adjusting the refractive power of a fluid-filled intraocular lens implanted into a patients eye, the method comprising:
selecting a pattern to cause a flattening of the intraocular lens or an increase in curvature of the intraocular lens; and
ablating the pattern onto a portion of the intraocular lens to alter either one or both of a refractive power and an amplitude of accommodation of the intraocular lens,
wherein the step of ablating the pattern onto the portion of the intraocular lens occurs while the intraocular lens remains implanted in the patients eye;
wherein the step of ablating the pattern onto the portion of the intraocular lens maintains the integrity of a fluid-filled interior cavity defined between the optical element and the flexible element;
wherein the step of ablating the pattern onto the portion of the intraocular lens causes the flattening of the intraocular lens or the increase in curvature of the intraocular lens; and
wherein the portion of the intraocular is an optical element of the intraocular lens or a flexible element of the intraocular lens.

2. The method of claim 1, wherein the portion of the intraocular lens is ablated by an energy source, focused or diffused.

3. The method of claim 2, wherein the energy source is a laser.

4. The method of claim 3, wherein the laser is selected from the group consisting of a YAG laser and a femtosecond laser.

5. The method of claim 4, wherein the portion of the intraocular lens is ablated to thin a surface of the intraocular lens to provide a greater amplitude of accommodation of the intraocular lens.

6. The method of claim 4, wherein the pattern comprises a circular region on the portion of the intraocular lens.

7. The method of claim 4, wherein the pattern comprises a ring-shaped region on the portion of the intraocular lens.

8. The method of claim 4, wherein the pattern comprises arcuate ablations.

9. The method of claim 8, wherein the arcuate ablations correct an astigmatism of the patients eye.

10. The method of claim 4, wherein the pattern is selected to cause the flattening of the intraocular lens.

11. The method of claim 4, wherein the pattern is selected to cause the increase in curvature of the intraocular lens.

12. The method of claim 1, wherein the ablating is performed within an optical axis of the patient's eye.

13. The method of claim 1, wherein the ablating is performed entirely outside of the optical axis of the patient's eye.

14. The method of claim 1, wherein a surface is disposed on one or more haptics associated with the fluid-filled intraocular lens.

15. The method of claim 14, further comprising the step of ablating a groove onto the surface of one or more haptics.

16. A method for adjusting the refractive power of a fluid-filled intraocular lens implanted into a patient's eye, the method comprising:
ablating a portion on either one or both of an anterior region and/or a posterior region of the implanted fluid-filled intraocular lens;
wherein the ablating maintains the integrity of a fluid-filled cavity defined between the anterior region and the posterior region of the fluid-filled intraocular lens.

17. The method of claim 15, wherein the ablated portion is on a surface of either one or both of the anterior and posterior portions.

18. The method of claim 17, wherein the ablating removes less than about 50% of a thickness of the surface.

19. The method of claim 15, wherein the ablated portion is disposed within a thickness of either one or both of the anterior and posterior portions.

20. The method of claim 19, wherein the ablated portion results in the creation of a hollow cavity.
